(19) United States
(12) Patent Application Publication
(10) Pub. No.: US 2015/0290226 A1
(43) Pub. Date: Oct. 15, 2015

(54) NOVEL LYOPHILIZED COMPOSITIONS OF CYCLOPHOSPHAMIDE

(71) Applicant: LEIUTIS PHARMACEUTICALS PVT. LTD., Andhra Pradesh (IN)

(72) Inventors: Kocherlakota Chandrashekhar, Secunderabad (IN); Banda Nagaraju, Hyderabad (IN)

(21) Appl. No.: 14/439,079

(22) PCT Filed: Jul. 29, 2013
(86) PCT No.: PCT/IN2013/000469

§ 371 Date: Apr. 28, 2015

(30) Foreign Application Priority Data
Oct. 29, 2012 (IN) 4483/CHE/2012

(51) Int. Cl. A61K 31/675; A61K 9/19

(52) U.S. Cl. CPC A61K 31/675; A61K 9/19

(57) ABSTRACT

The present invention relates to the process for producing novel lyophilized compositions of Cyclophosphamide, wherein the process does not need rehydration step. The compositions of the present invention have greater stability and uniformity.

### CROSS-REFERENCE TO RELATED APPLICATIONS

This application claims priority to PCT Application No. PCT/IN2013/000469, having a filing date of Jul. 29, 2013, based on IN Application No. 4483/CHE/2012, having a filing date of Oct. 29, 2012, the entire contents of which are hereby incorporated by reference.

### FIELD OF TECHNOLOGY

Cyclophosphamide is a synthetic antineoplastic drug chemically related to the nitrogen mustards and has the following structure:

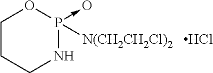

The chemical name of cyclophosphamide is 2-[bis(2-chloroethyl)amino]tetrahydro-2H-1,3,2-oxazaphosphorine 2-oxide monohydrate. The compound along with related novel cyclic phosphoric acid ester amides was disclosed and claimed in U.S. Pat. No. 3,018,302.

### BACKGROUND

Cyclophosphamide comprises of monohydrate and anhydrous forms. The monohydrate is a stable form but under dry conditions (relative humidities of about 20% or less) the monohydrate begins to lose this water of hydration which can cause problems in manufacture. Hence maintaining the manufacturing and storage temperatures for this product is extremely important.

U.S. Pat. No. 4,659,699 to Daniel et al. discloses the process for freeze drying of Cyclophosphamide. The two stage process described in the patent involves freeze drying of an aqueous solution of Cyclophosphamide to yield a hydrate of Cyclophosphamide. In the first stage, Cyclophosphamide is freeze dried with an excipient until the moisture content is less than 2% by weight. In the second stage, the freeze dried material is rehydrated until the moisture content of the product is in the critical range i.e 2-7% by weight. The process described in this patent requires the use of high quantity of excipients for maintaining the stability of the product. U.S. Pat. No. 4,537,883 to Alexander et al. discloses various lyophilizates of Cyclophosphamide. These lyophilizates are prepared by lyophilizing a solution of Cyclophosphamide and one or more excipients and rehydrating the product such that it contains about 4% moisture. The lyophilizates described in this patent used various excipients like mannitol, sodium bicarbonate, lactose, polyvinyl pyrrolidone (PVP), arginine, and tartaric acid.

U.S. Pat. No. 4,537,883 to Nageswara R. Palepu et al. discloses a stable rapidly dissolving lyophilized and hydrated composition of Cyclophosphamide with sodium bicarbonate. The disadvantages associated with the product described in this patent are the large size of the vials required for lyophilization and time taken to solubilise the product.

Cyclophosphamide is available as monohydrate in parenteral dosage formulation consisting of sterile packaged dry powder blend mixtures of drug and sodium chloride. The premixes were dissolved in water prior to administration. During the processing and storage of dry powder premix formulation, a glassiness and or stickiness could be acquired by the premix composition giving unattractive material with inferior solubility characteristics and decreased potency.

Hence there is a need to develop formulations of Cyclophosphamide overcoming the disadvantages of products and processes known in the art.

### SUMMARY

An aspect relates to developing lyophilized formulations of Cyclophosphamide using a process that does not need rehydration step.

Another aspect is to describe process for producing a stable lyophilized cyclophosphamide monohydrate formulation by using a suitable solvent or mixture of solvents in suitable proportions and optionally contains other excipients.

Another aspect is to develop formulations of Cyclophosphamide monohydrate with uniform hydrate integrity between the vials.

Another aspect is to provide Cyclophosphamide formulations with improved stability and lesser time for reconstitution.

It is yet another aspect to provide a stability indicating analytical method to monitor drug product quality.

### BRIEF DESCRIPTION

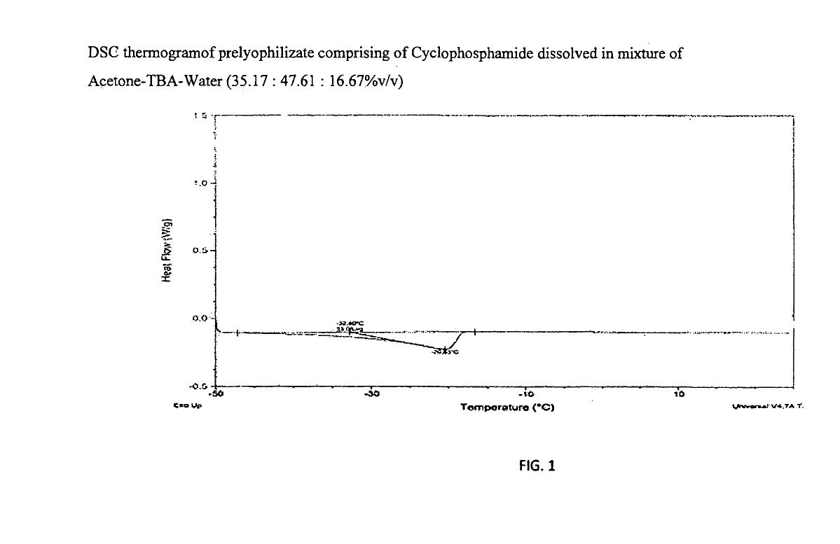

FIG. 1 shows the DSC thermogram of prelyophilizate comprising of Cyclophosphamide dissolved in mixture of Acetone-TBA-Water (35.17:47.61:16.67% v/v);

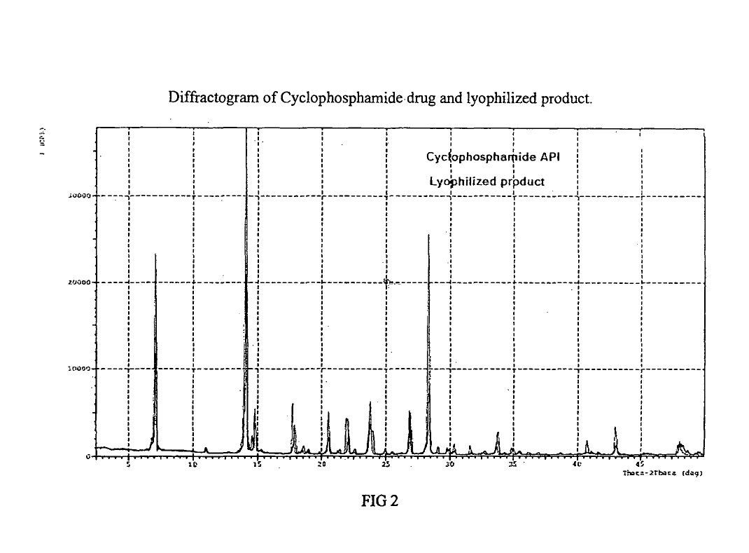

FIG. 2 depicts Diffractogram of Cyclophosphamide drug and lyophilized product;

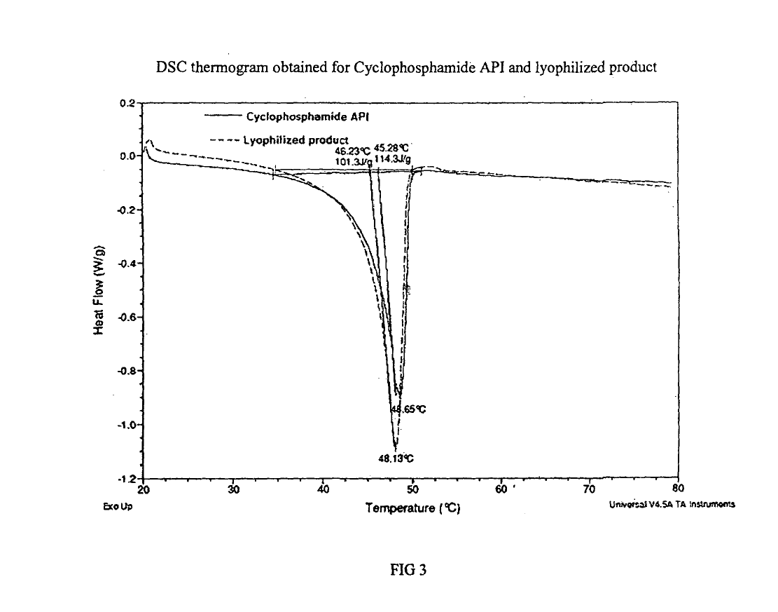

FIG. 3 depicts a DSC thermogram obtained for Cyclophosphamide drug and lyophilized product and

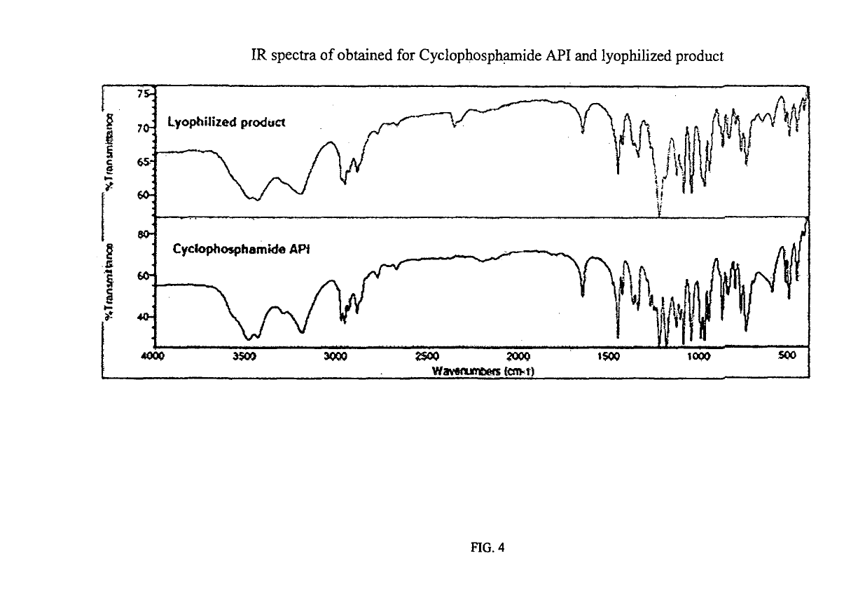

FIG. 4 depicts an IR spectra obtained for Cyclophosphamide drug and lyophilized product.

### DETAILED DESCRIPTION

The objective of the present invention is to prepare a stable lyophilized parenteral pharmaceutical compositions of cyclophosphamide monohydrate.

An aspect of the present invention relates to the process for producing a stable lyophilized cyclophosphamide monohydrate formulation by using a suitable solvent or mixture of solvents in suitable proportions and optionally contains other excipients.

Another aspect of the present invention is to describe lyophilization process for producing pharmaceutical compositions of cyclophosphamide monohydrate. A lyophilization process of cyclophosphamide injection comprises of following steps: (a) Dissolving or dispersing cyclophosphamide in organic solvents or mixtures thereof (b) Filling the solution or dispersion in vials (c) Freezing the solution or dispersion (d) drying.

Another aspect of the present invention is to develop formulations of Cyclophosphamide monohydrate with uniform hydrate integrity between the vials and improved stability and lesser time for reconstitution.

Another aspect of the present invention is to describe stability indicating analytical method to monitor drug product quality.

The invention involves forming stable monohydrate “in situ” during lyophilization without the need for a rehydration step as taught in the prior art. This aspect of the invention is achieved by (i) a proper selection of solvent system and/or (ii) optimal process conditions during the lyophilization process.

The present invention discloses a process for manufacturing a lyophilized preparation of cyclophosphamide intended for use in parenteral administration. The pharmaceutical formulation of the invention comprises Cyclophosphamide monohydrate and at least one organic solvent or mixture of solvents to dissolve the drug. The solvent is later removed during the freeze drying process. The formulation additionally comprises water to facilitate the formation of the stable monohydrate form during lyophilization.

Freeze drying process involves removal of solvent from a frozen mass under reduced atmospheric pressure. In the context of this invention the term freeze drying, drying and Lyophilization shall be used interchangeably. Lyophilization helps stabilize pharmaceutical formulations by reducing the solvent component or components to levels that no longer support chemical reactions or biological growth. Since drying during lyophilization takes place at a low temperature, chemical decomposition is also reduced.

Annealing shall be defined as process of transient increase in product temperature from initial set point to higher or lower set point, and then bringing the product temperature back to original set point. Annealing can be done on product during different steps of freeze drying process.

Frozen mass shall be defined as a product kept at temperature where in the physical state of the product changes from liquid to semi solid mass or solid mass. In the context of the present invention a frozen mass shall also mean a product kept at a temperature lower than zero degrees centigrade.

Prelyophilizate shall mean a composition comprising cyclophosphamide dissolved or suspended in solvents and meant to be subjected to freeze drying. The prelyophilizate may comprise suitable pharmaceutically acceptable excipient selected from list of excipients as described in the present invention. The excipients or drug shall be in dissolved or suspended form in the solvent mixture.

As used herein, the term “freeze-dried formulation” or “Lyophilizate” shall be defined as a dried product obtained as a result of freeze drying or lyophilization or drying process using a freeze dryer.

Freeze drying apparatus comprises of a chamber, condenser, and vacuum system with programmable features to control temperature and vacuum during drying process. The equipment optionally may have solvent trap.

The injectable formulations of the present invention comprises pharmaceutically acceptable carrier. The pharmaceutically acceptable carrier or excipient can be selected from solvent, bulking agent, complexing agents, preservatives, anti-oxidants, stabilizers, tonicity modifiers or any other suitable adjuvant thereof

As used herein the term “solvent” refers to the liquid component of a formulation that is capable of dissolving or suspending one or more solutes. The term “solvent” can refer to a single solvent or a mixture of solvents. The solvent, as mentioned, can be any liquid in which the material dissolves or could be suspended; the solvent can be a single substance or a mixture of co-solvents. Depending on the formulation or the freeze-drying process, it may be desirable to include one or more organic solvents in the liquid formulation. Suitable solvents include the following, but are not limited to Acetone, Acetonitrile, Tertiary butanol, Methanol, Ethanol, Isopropyl alcohol, Ethylacetate, Toulene, Propyl acetate, Nitromethane, 1,4-Dioxane, Heptane, Methylcyclohexane, Formic acid, isopropyl acetate, 1,2-Dimethoxyethane, 1,1,2-Trichloroethene, Methylethyl ketone, Cyclohexane, Ethyl formate, Hexane, Tetrahydrofuran, Chloroform, Methyl acetate, 1,2-Dichloroethene, tert-Butylmethyl ether, Dichloromethane, Pentane, Ethyl ether, Sulfalone, Ethyleneglycol, Formamide, Tetralin, N-Methylpyrrolidone, Dimethyl sulfoxide, N,N-Dimethylacetamide, 1-Pentanol, 3-Methyl-1-butanol, Anisole, N,N-Dimethylformamide, 2-Ethoxyethanol, 1-Butanol, 2-Methoxyethanol, Cumene, Butyl acetate, 2-Methyl-1-propanol, Chlorobenzene, Acetic acid, 2-Butanol, Isobutyl acetate, 1-Propanol, Pyridine, Methylbutyl ketone, Methyl isobutyl ketone, Xylene, Dimethyl carbonate, Hexafluoroacetone, Chlorobutanol, Dimethylsulfone, carbon tetrachloride, tetrahydropyran, dioxane, trioxane and other cyclic mono-, di- and tri-ethers, propylene glycol (PG), polyethylene glycol, glycerine. The solvent system may additionally comprise water. Mixtures of solvents selected are used in a suitable proportion and suitable quantity to achieve desirable effect.

The purpose of the bulking agent is to provide bulk to the formulation and enhance cake formation. Bulking agents include saccharides, preferably monosaccharides or oligosaccharides, sugar alcohols, and mixtures thereof. Suitable bulking agents include the following, but are not limited to mannitol, sodium chloride, glucose, sucrose, lactose, trehalose, dextrose, maltose, sorbitol, dextran, raffinose, PVP, histidine, amino acids such as glycine, arginine, aspartic acid and the like.

Tonicity modifier can also be optionally added to the formulation. Suitable tonicity modifiers include the following, but are not limited to mannitol, dextrose, sucrose, glycine, glycerol, sodium chloride and the like.

Stabilizing agents are typically added to a formulation to improve stability of the formulation. Suitable examples of stabilizing agents include cryoprotectants, lyoprotectants, crystallization inhibitors or any other suitable stabilizer thereof. Suitable stabilizers include the following, but are not limited to Saccharides, including monosaccharides such as glucose, disaccharides such as sucrose (glucose+fructose), lactose (glucose+galactose), maltose (glucose+glucose), and trehalose (alpha-D-glucopyranosyl alpha-D-glucopyranoside), and polysaccharides such as dextran (polysaccharide containing glucose monomers, Crystallization inhibitors such as PVP (polyvinylpyrrolidone), HPC (hydroxypropyl cellulose), or HPMC (hydroxypropylmethylcellulose) and the like can be used. Surfactants also act as suitable stabilizers such as polyoxyethylene sorbitan monolaurate (Tween™ 20, Tween™ 80), pluronic F-68, Triton™ X-100, and sodium dodecyl sulfate (SDS), polysorbate or any other suitable surfactant can be selected.

Buffers are typically included in pharmaceutical formulations to maintain the pH of the formulation at a physiologically acceptable pH. The desirable pH for a formulation may also be affected by the active agent. Examples of suitable buffers include buffers derived from an acid such as phosphate, aconitic, citric, gluaric, malic, succinic and carbonic acid, alkali or alkaline earth salt of one of these acids, Tris buffer, histidine buffers, meglumine or any suitable buffer thereof pH adjusting agents such as, but are not limited to sodium hydroxide, sodium carbonate, sodium bicarbonate, potassium hydroxide, ammonium carbonate, hydrochloric acid, citric acid, lactic acid, phosphoric acid, sodium phosphate, sulfuric acid, and the like can also be used.

Other excipients may also be added to the lyophilized preparations of the present inventions. Such excipients may include antioxidants, antimicrobials, cryostabilizers, or any other suitable pharmaceutically acceptable adjuvants thereof

The lyophilized product or the dried product shall be reconstituted and further diluted using suitable diluents. Suitable diluents may include the following but are not limited to water, sodium chloride, dextrose, sugars, polyols, electrolytes or any other parenterally acceptable diluents and mixtures thereof

Selection of Solvent System and Ratios

The solubility of Cyclophosphamide monohydrate in various solvents was checked. The quantity of solvent consumed for dissolving 100 mg of Cyclophosphamide monohydrate is given below:

| | |
|---|---|
| Water | 2.45 ml |
| Acetonitrile | 0.5 ml |
| Acetone | 0.4 ml |
| Ethanol | 0.5 ml |
| Tertiary butyl alcohol | 0.5 ml |
| Dimethyacetamide | 2.0 ml |

Lyophilization of Cyclophosphamide with Different Solvent Systems

The investigators found surprising results that Cyclophosphamide monohydrate was obtained without rehydration step when Cyclophosphamide was freeze dried in presence of solvents.

Binary solvent mixtures were evaluated for lyophilization of Cyclophosphamide for injection.

Solvent systems used for the study are given below in Table-1:

TABLE 1
Binary solvent Mixtures used for Lyophilization

| | | Solvent mixture Composition (% v/v) | | | | |
|---|---|---|---|---|---|---|
| | Qty of API | | | Tertiary | | Total Fill |
| S.No | per vial | Acetone | Acetonitrile | Butanol | Water | Volume mL |
| 1 | 2 gms | NA | 70 | NA | 30 | 8.40 mL |
| 2 | 1 gm | 50 | NA | NA | 50 | 5.50 mL |
| 3 | 2 gms | NA | NA | 96 | 4 | 7.65 mL |

NA: Not Applicable

The above mentioned solvent mixture ratios were lyophilized without rehydration step and the results of lyophilizate are given below

TABLE 2
Results of Binary solvent Mixtures used for Lyophilization

| | Qty of | | | | | Results | | | |
|---|---|---|---|---|---|---|---|---|---|
| | API | Solvent mixture Composition (% v/v) | | | | Water | Residual Solvent (ppm) | | |
| | per | | | Tertiary | | Content | | | Tertiary |
| S.No | vial | Acetone | Acetonitrile | Butanol | Water | (% w/w) | Acetone | Acetonitrile | Butanol |
| 1 | 2 gms | NA | 70 | NA | 30 | 5.80 | NA | 2500 | NA |
| 2 | 1 gms | 50 | NA | NA | 50 | 6.29 | 5838 | NA | NA |
| 3 | 2 gms | NA | NA | 96 | 4 | 6.64 | NA | NA | 2768 |

Observations & Conclusion:

From the analytical data it was observed that the water content was found to be within the acceptable range for monohydrate (5.7% w/w to 7.5% w/w) whereas the solvent levels are elevated.

Freeze Drying of Cyclophosphamide Using Mixture of Solvents:

Solvent mixtures of Acetone-Acetonitrile-Water and Acetone-Tertiary Butyl Alcohol-Water were evaluated to establish the suitability in lyophilization. The results are tabulated in table 3.

TABLE 3
Results after lyophilization with Acetone-Acetonitrile-Water and Acetone-TBA-Water:

| | Qty of | Solvent mixture Composition (% v/v) | | | | | Results | | | |
|---|---|---|---|---|---|---|---|---|---|---|
| | API | | | | | Total | Water | | | |
| | per | | | | | volume | Content | Residual Solvent Content (ppm) | | |
| S.No | vial | Acetone | Acetonitrile | Water | TBA | (mL) | % (w/w) | Acetone | Acetonitrile | TBA |
| 1 | 2 gms | 40 | 10 | 50 | NA | 11 | 21.91 | 3974 | 968 | NA |
| 2 | 2 gms | 28.57 | NA | 33.33 | 38.09 | 11.3 | 36.38 | 1015 | NA | 514 |

*Not applicable

From the analytical results it is evident that Cyclophosphamide when freeze dried using mixture of Acetone: Acetonitrile: Water solvent mixture resulted in high residual solvent content and water content whereas the solvent system containing Acetone: TBA: Water showed low residual solvent content and significantly high water content.

The proportions of constituents of the solvent mixture was optimized by varying the % of water in the solvent system varying from about 7.8% v/v to 16.6% v/v to evaluate the effect of water on the drying Acetone, TBA and Water. The results are tabulated in table 4:

TABLE 4
Results of solvent mixture proportions on the lyophilizate:

| | Qty of | | | | | Results | | |
|---|---|---|---|---|---|---|---|---|
| | API | | | | Total | Water | Residual Solvent | |
| | per | Solvent mixture Composition (% v/v) | | | volume | Content | Content (ppm) | |
| S.No | vial | Acetone | TBA | Water | (mL) | % (w/w) | Acetone | TBA |
| 1 | 2 gms | 39.47 | 52.63 | 7.89 | 9.0 | 6.58 | 6861 | 1601 |
| 2 | 2 gms | 37.5 | 50 | 12.5 | 9.4 | 6.57 | 1234 | 557 |
| 3 | 2 gms | 35.72 | 47.62 | 16.66 | 9.9 | 6.53 | 1018 | 661 |

The data in the above table shows that the level of residual solvent content in the lyophilizate was reduced by varying the proportions of water in the solvent mixture.

The impact of constituents of solvent composition used to dissolve cyclophosphamide was assessed by varying Acetone, TBA and Water in the pre-lyophilizate composition. The results are in the table 5.

TABLE 5
Effect of solvent composition used to dissolve Cyclophosphamide on lyophilizate:

| | Qty of | | | | | | | Results | | |
|---|---|---|---|---|---|---|---|---|---|---|
| | API | | Solvent mixture | | | Total | Lyophilization | Water | Residual Solvent | |
| | per | | Composition (% v/v) | | | Volume | Cycle Time | Content | content ppm | |
| S.No | vial | | Acetone | TBA | Water | (mL) | (Hrs) | (% w/w) | Acetone | TBA |
| 1 | 500 | mg | 57.14 | 42.85 | 0.00 | 2.1 | 68.2 | 6.37 | 8312 | 2388 |
| 2 | 2 | gms | 41.66 | 55.55 | 2.77 | 8.6 | 78.7 | 6.45 | 1640 | 1046 |
| 3 | 2 | gms | 39.47 | 52.63 | 7.89 | 9.0 | 71.0 | 6.58 | 6861 | 1601 |
| 4 | 2 | gms | 37.5 | 50.0 | 12.5 | 9.4 | 71.0 | 6.57 | 1234 | 557 |
| 5 | 2 | gms | 35.17 | 47.61 | 16.66 | 9.9 | 71.0 | 6.53 | 1018 | 661 |
| 6 | 2 | gms | 34.88 | 46.51 | 18.60 | 10.1 | 78.7 | 6.37 | 792 | 538 |
| 7 | 2 | gms | 34.09 | 45.45 | 20.45 | 10.3 | 78.7 | 6.36 | 1575 | 428 |
| 8 | 2 | gms | 33.33 | 44.44 | 22.22 | 10.5 | 78.7 | 6.32 | 1730 | 387 |
| 9 | 2 | gms | 32.60 | 43.47 | 23.91 | 10.7 | 78.7 | 6.46 | 2010 | 437 |
| 10 | 2 | gms | 25.00 | 33.33 | 41.66 | 13.0 | 75.2 | 6.57 | 1122 | 706 |

From above it is evident that residual solvent content, and target water content in the lyophilizate are within acceptable range.

Optimisation of process conditions Cyclophosphamide is stable in its crystalline monohydrate form. The change of description of Cyclophosphamide monohydrate at various temperatures was studied by exposing the drug to different temperatures under constant vacuum and details are given in Tables 6 & 7:

TABLE 6
Description of Cyclophosphamide monohydrate at different
temperatures at 1000 mtorr process vacuum:

| | | Vacuum (1000 mtorr) | | | | |
|---|---|---|---|---|---|---|
| | | +25° C. | −2° C. | −15° C. | −25° C. | −40° C. |
| Condition | Initial | (5 hrs) | (12 hrs) | (19 hrs) | (12 hrs) | (5 hrs) |
| % Moisture | 6.87 | 2.94 | 4.52 | 6.29 | 6.27 | 6.23 |
| content | | | | | | |
| Observation | Powder | Liquid | Sticky | Powder | Powder | Powder |
| for | | | mass | | | |
| Description | | | | | | |

TABLE 7
Description of Cyclophosphamide monohydrate
at different temperatures and process vacuum:

| | | | Observation/Result | |
|---|---|---|---|---|
| | Temperature | Vacuum (mtorr) | | Moisture |
| S. No. | (° C.) | and duration | Description | Content (%) |
| 1 | −10 | 350 mtorr and | Powder | 7.56% |
| | | 6 hours | | |
| 2 | −15 | 350 mtorr and | Powder | 6.99% |
| | | 12 hours | | |
| 3 | −25 | 150 mtorr and | Powder | 6.87% |
| | | 6 hours | | |
| 4 | −25 | 150 mtorr and | Sticky Mass | 4.55% |
| | | 15 hours | | |
| 5 | −25 | 280 mtorr and | Powder | 6.51% |
| | | 12 hours | | |

The results indicate that physical stability or description of Cyclophosphamide is susceptible to temperature and vacuum. At higher temperatures and vacuum, Cyclophosphamide monohydrate loses water and melts. When it starts losing its water molecule, the physical description changes from powder to sticky mass or liquid, hence selection of the process conditions (like temperature, vacuum etc) is critical for lyophilization process so as to obtain monohydrate form. The selection of process conditions that result in insitu formation of monohydrate form of Cyclophosphamide are also dependent on composition of pre-lyophilizate.

The formulation of the present invention preferably has a solvent or mixture of solvents. The inventors have surprisingly found by use of mixture of solvents with or without water, or mixture of solvent with water yielded best results.

Evaluation of Process Conditions for Drying

The drug solution was transferred into sample pan and sealed with lid. DSC Q2000 was purged with nitrogen. Sample was cooled from 25° C. to −50° C. and heated from −50° C. to 25° C. at the rate of 1.5° C./min.

From the DSC thermogram of pre-lyophilizate as shown in FIG. 1 it was observed that melt of frozen mass started at −32.80° C. and complete melt was observed at −20.53° C. So, to enhance the crystallization, for controlled removal of water and to produce a uniform hydrate insitu during the process, the temperature of product during the drying process was kept on hold for −18° C. for removal of water and TBA. Duration of hold at −18° C. and 600 mtorr is important for insitu formation of Cyclophosphamide monohydrate and effective removal of excess TBA. This was evaluated by varying the duration of hold at −18° C. at 600 mtorr pressure during drying process as given in table 8:

TABLE 8
Analysis results of lyophilizate by varying the duration of hold at −18° C., 600 mtorr:

| | | | | | Duration | | | |
|---|---|---|---|---|---|---|---|---|
| | Qty of | | | | of Hold of | Analysis results of lyophilizate | | |
| | API | Solvent mixture | | | −18° C. 600 | Water | Residual solvent | |
| | per | Composition (% v/v)* | | | mtorr | Content | content (ppm) | |
| S.No | vial | Acetone | TBA | Water | (min) | (% w/w) | Acetone | TBA |
| 1 | 2 gms | 35.71 | 47.62 | 16.67 | 680 | 6.53 | 1018 | 661 |
| 2 | 2 gms | 35.71 | 47.62 | 16.67 | 1200 | 6.56 | 857 | 621 |
| 3 | 2 gms | 35.71 | 47.62 | 16.67 | 3000 | 6.88 | 848 | 505 |

*Note: The total volume in all of above compositions was 9.9 mL

The results showed that residual solvent content reduced in the lyophilizate with increase in hold time while retaining the water content of insitu monohydrate obtained.

Evaluation of Lyophilizate:

The monohydrate form present in the lyophilizate was evaluated in comparison with drug substance used in the process by way of following tests:

Moisture Content and Water Activity:

The moisture content and water activity of the drug substance as well as the lyophilizate was checked to ensure the retention of the crystalline monohydrate form of the drug substance in the Lyophilizate. Water activity instruments measure the amount of free (sometimes referred to as unbound or active) water present in the sample and KF titration measures the total water in a sample. The values are tabulated in tables 9 and 10.

TABLE 9
Water content results of Drug substance and Lyophilizate

| | |
|---|---|
| Sample | Moisture Content (%) |

| number | Drug substance (Monohydrate Form) | Lyophilizate |
|---|---|---|
| I | 6.64 | 5.87 |
| II | 6.75 | 6.43 |
| III | 6.72 | 6.07 |

TABLE 10
Water activity results for Drug substance and Lyophilizate

| | |
|---|---|
| Sample | Water Activity (a_{w}) |

| number | Drug substance (Monohydrate Form) | Lyophilizate |
|---|---|---|
| I | 0.374 | 0.375 |
| II | 0.372 | 0.383 |
| III | 0.373 | 0.386 |

Results for moisture content as well as the water activity were similar for the drug substance as well as the lyophilizate confirming that the crystalline monohydrate form of the API was retained in the lyophilized product. Crystalline monohydrate form contains 5.7 to 6.8% of water which is in agreement with the results obtained in table 6 to confirm that the lyophilizate is a stable monohydrate.

X-Ray Diffraction Studies:

XRD was performed using Schimadzu XRD-7000 at following conditions:

### X-ray Tube: Cu (1.54060 A)

### Voltage: 45.0 kV

### Current: 40.0 mA

### Scan Range: 2.5000<->49.9980 deg

### Step Size: 0.0170 deg

### Count Time: 0.51 sec

### Slit DS: 1.00 deg

### SS: 1.00 deg

### RS: 0.15 mm

The diffractograms of drug substance and lyophilizate as shown in FIG. 2; are comparable confirming that lyophilizate is a monohydrate form.

DSC Studies

DSC was performed using TA-Q20. 3.5 mg of sample was accurately weighed into aluminium pan. The pan was crimped and DSC was performed at a heating rate of 2° C./min from 25° C. to 80° C. under atmosphere of nitrogen.
The DSC thermograms of drug substance and lyophilizate are similar as shown in FIG. 3.

IR Studies:

1 to 2 mg of sample being examined was triturated with 300 mg to 400 mg of dried Potassium bromide. The sample was then scanned using a FT-IR spectrophotometer (Nicolet IS, Thermoscientific).

The IR spectra of drug substance and lyophilizate are similar as shown in FIG. 4.

Analytical Evaluations

Lyophilized product was analyzed by HPLC for % purity and Assay. The result shows that the sample contains 0.27% impurities and was found to be 99.73% pure. By Assay HPLC was found to be 101% which is similar to the bulk solution assay prior to the lyophilization. These results show that during the lyophilization process sample was not degraded and crystalline monohydrate form is retained in final product.

Cyclophosphamide monohydrate is official in USP and Ph.Eur. Both the monographs specify degradation analytical methods by Thin layer Chromatography. The TLC methods are time consuming and require usage of expensive USP reference impurity standards for comparison of Spots on TLC. These methods are not suitable for estimation of other degradation products or any source of contamination during manufacturing and storage of drug product.

Thus there is a need to develop stability indicating HPLC methods for monitoring quality of drug substance and drug product throughout the shelf life. The investigators developed novel HPLC analytical methods where in the polarity of mobile phase was gradually changed in order to separate the major degradation products on single Chromatographic run.

TABLE 11
The impurities in Cyclophosphamide and separation techniques are as
follows:

| Name of | | Molecular | Molecular | | Analytical |
|---|---|---|---|---|---|
| Impurity | Chemical name | Formula | weight | Structure | method |
| Impurity- A | N,N-Bis(2- chloroethyl)amine hydrochloride | C₄H₁₀Cl₃N | 178.49 | 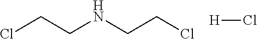 | HPLC method |
| Impurity- B | 3-(2-Chloroethyl)- 2-oxo-2-hydroxy- 1,3,6,2- oxadiazaphosphonane | C₇H₁₆ClN₂O₃P | 242.64 | 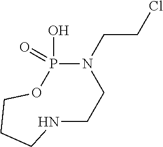 [SMILES: O=P1(O)OCCCNCCN1CCCl] | HPLC method |
| Impurity- C | 3-Aminopropyl 17dihydrogen phosphate | C₃H₁₀NO₄P | 155.09 | 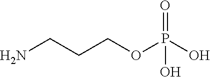 | HPLC with post column dervitization |
| Impurity- D | 3-((2-((2- chloroethyl)amino) ethyl)amino)propyl 17ihydrogen phosphate | C₇H₂₀Cₗ₃N₂O₄P | 333.58 | 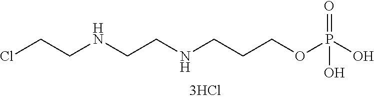 [SMILES: O=P(O)(O)OCCCNCCNCCCl] | HPLC method |

HPLC Method for Estimation of Impurity-A:

Mobile Phase-A: 0.5 ml of Triethylamine is diluted with in 1000 ml of HPLC grade (Ultrapure) water and pH adjusted to 7.5 with dilute Orthophosphoric acid.
Mobile Phase-B: Mobile phase-A and Acetonitrile are mixed in the ratio of (30:70) % v/v and degassed in a sonicator for about 10 min.

Preparation of Diluent: Mobile Phase-A is used as diluent and maintained at temperature about 2-8° C. in the refrigerator.

Typical Chromatographic Conditions:

Column: Kinetex C-18 (100 mm×4.6 mm), 2.6 μm
Wave length: 195 nm
Flow rate: 0.5 mL/min
Column oven Temperature: 25±2° C.
Sample cooling rack: 5±1° C.
Injection volume: 100 μL
Runtime: 65 minutes

### Gradient Program:

TABLE 12
chromatographic conditions for detection of Impurity A

| Time(min) | % Mobile Phase-A | % Mobile Phase-B |
|---|---|---|
| 0 | 100 | 0 |
| 20 | 85 | 15 |
| 40 | 40 | 60 |
| 50 | 40 | 60 |
| 51 | 100 | 0 |
| 65 | 100 | 0 |

Preparation of Standard Solution:

Preparation of Impurity-A solution: 0.2% Level 4 mg of Impurity-A was weighed into a 100 ml volumetric flask and 50 ml of diluent was added and sonicated for dissolution. The volume was made up with the diluent.

Preparation of Sample Solution: (20 mg/ml)

100 mg of Cyclophosphamide was weighed into a 5 ml volumetric flask and 3 ml of diluent was added and sonicated. Volume was made up with the diluent.

Procedure:

Injection-Blank (as diluent), diluted standard for two times and test preparation in single into the liquid chromatographic system.

System Suitability Parameters:
The tailing factor for Impurity-A in standard solution is not more than 2.0
The USP theoretical plates for Impurity-A is not less than 2000
The relative standard deviation for two replicate injections is not more than 10.0%

Detection of Impurity B and D

Mobile Phase-A: 1 ml of Orthophosphoric acid is mixed in 1000 ml of water (0.1%) and degassed in sonicator for about 10 min.

Mobile Phase-B: Water and Acetonitrile were mixed in the ratio of 20:80% v/v and degassed in a sonicator for about 10 min.

Preparation of Diluent: HPLC grade (Ultrapure) water maintained at temperature about 2-8° C.

Typical Chromatographic Conditions:

Column: Inertsil-ODS 3, 25 cm×4.6 mm, 5 μm (L1)

Wave length: 195 nm

Flow rate: 0.5 mL/min

Column oven Temperature: 25±2° C.

Sample cooling rack: 5±1° C.

Injection volume: 100 μL

Runtime: 75 minutes

Sample concentration: 20 mg/mL

Gradient Program:

TABLE 13
chromatographic conditions for detection of Impurity B and D

| Time | % Mobile Phase-A | % Mobile Phase-B |
|---|---|---|
| 0 | 100 | 0 |
| 10 | 100 | 0 |
| 30 | 75 | 25 |
| 50 | 20 | 80 |
| 60 | 20 | 80 |
| 61 | 100 | 0 |
| 75 | 100 | 0 |

Preparation of Standard Solution: (0.2% Level)

40.0 mg of Cyclophosphamide working standard was transferred into a 100 ml volumetric flask. 50 ml of diluent was added sonicated and volume was made up with diluent. 1 ml of this solution was diluted to 10 ml with diluent.

Preparation of Sample:

100 mg sample was transferred into a 5 ml volumetric flask, 3 ml was added and sonicated and made to volume with diluent.

Procedure:

Injection—the Blank (as diluent), diluted standard for two replicate injections and a single test preparation into the liquid Chromatographic system.

### System Suitability Parameters:

1. The tailing factor for Cyclophosphamide peak in standard solution is not more than 2.0
2. The USP theoretical plates for Cyclophosphamide peak is not less than 4000
3. The relative standard deviation for two replicate injections is not more than 10.0%

Calculation:

Blank peaks are disregarded and all impurity peaks and Cyclophosphamide peaks are integrated. Peak below 0.05% is disregarded.

TABLE 14
Cyclophosphamide Impurities and their retention times

| S. | Name of the impurity | *RRT (about) | #RRF |
|---|---|---|---|
| 1. | Cyclophosphamide related compound D | 0.12 | 0.28 |
| 2. | Cyclophosphamide related compound B | 0.17 | 1.06 |
| 3. | Cyclophosphamide | 1.0 | 1.0 |

*RRT—Relative retention time
#RRF—Relative retention factor

Detection of Impurity C

Impurity-C cannot be detected in HPLC UV detector because the impurity-C does not contain the necessary chromophoric, fluophoric or redox groups. However, this problem was overcome by inducing derivatization reaction. In post-column mode, the reaction is performed automatically by adding a derivatization reagent after separation, and before detection, by means of a second HPLC pump.

Fluorescamine is used as dervitizing reagent for Impurity-C. The derivitized impurity-C was well detected at 380-nm.

Mobile Phase-A: 1.7121 g of di-sodium hydrogen phosphate (0.01M) was transferred in 1000 ml of HPLC grade (Ultrapure) water and mixed well, pH 10.5 adjusted with diluted Sodium hydroxide.

Mobile Phase-B: Acetonitrile and HPLC grade (Ultrapure) water were mixed in the ratio of (90:10) v/v and sonicated for 10 minutes for degassing.

Preparation of Diluent: HPLC grade (Ultrapure) water maintained at temperature about 2-8° C. is used as diluent.

Typical Chromatographic Conditions:

Column: Purospher Star RP18, (250 mm×4.6 mm) 5 μm (L1)

Wave length: 380 nm

Flow rate: 1.0 mL/min

Column oven Temperature: 25±2° C.

Sample cooling rack: 5±1° C.

Injection volume: 100μL

Runtime: 35 minutes

Sample concentration: 20 mg/mL

Gradient Program:

TABLE 15
Chromatographic conditions for detection of Impurity C

| Time | % Mobile Phase-A | % Mobile Phase-B |
|---|---|---|
| 0 | 100 | 0 |
| 6 | 100 | 0 |
| 10 | 20 | 80 |
| 25 | 20 | 80 |
| 30 | 100 | 0 |
| 35 | 100 | 0 |

External Pump Conditions:

### Connector: ‘T’ Joint

Flow rate: 1.0 ml/min

Mobile Phase for external pump: Accurately weighed 80 mg of Fluroscamine derivitizating reagent was transferred to a 200 ml volumetric flask and 100 ml of Acetonitrile was added and the volume was made up volume with Acetonitrile and mixed well.

Preparation of Impurity-C: (≈10 ppm)-0.06% Level

About 5 mg of Cyclophosphamide Impurity-C was accurately weighed and transferred into a 10 ml volumetric flask and about 5 ml of water was added. The contents were sonicated and the volume was made up with water and further mixed well. Subsequently, 1 ml of this solution was diluted to 50 ml with water and mixed well.

Procedure for post column derivatization technique: A ‘T’ joint was connected to the column outlet of external pump and the other outlet of the column was connected to the HPLC detector. The mobile phase from HPLC pump and the mobile phase from external pump were mixed at the ‘T’ Joint connector and then pumped to the detector.

The following examples further describe certain specific aspects and embodiments of the present invention and demonstrate the practice and advantages thereof. It is to be understood that the examples are given by way of illustration only and are not intended to limit the scope of the invention in any manner.

### EXAMPLES

### Example-1

| | | Quantity | | Quantity | Quantity | Quantity |
|---|---|---|---|---|---|---|
| S. No | Ingredients | per ml | | mg/Vial | mg/Vial | mg/Vial |
| 1. | Cyclophos- | 202.02 | mg | 500 | 1000 | 2000 |
| | phamide | | | | | |
| 2. | Acetone | 0.303 | mL | 0.75 mL | 1.5 mL | 3 mL |
| 3. | Tertiary | 0.404 | mL | 1 mL | 2 mL | 4 mL |
| | butanol | | | | | |
| 4. | Water | 0.141 | mL | 0.35 mL | 0.70 mL | 1.4 mL |

The solvent mixture was prepared by taking acetone and TBA in a mixing vessel and the solution was cooled to 2-8° C. Cyclophosphamide was added to 80% of solvent mixture and stirred till completely dissolved followed by addition of water at 2-8° C. Volume was made up to 100% with solvent mixture and the bulk solution was filled into vials for lyophilisation. The lyophilization cycle parameters are mentioned in table 16.

TABLE 16
Freeze drying cycle process parameters:

| | Lyophi- | | Process | | | Cumulative |
|---|---|---|---|---|---|---|
| | lization | | Time | Ramp/ | Vacuum | time |
| Step # | Step | Temp ° C. | (min) | Hold | mtorr | (hrs) |
| 1 | Freezing | −5 | 10 | H | — | 0.166 |
| 2 | Freezing | −45 | 60 | R | — | 1.2 |
| 3 | Freezing | −45 | 180 | H | — | 4.2 |
| 4 | Freezing | −12 | 150 | R | — | 6.7 |
| 5 | Freezing | −12 | 300 | H | — | 11.7 |
| 6 | Freezing | −45 | 60 | R | — | 12.7 |
| 7 | Freezing | −45 | 180 | H | — | 15.7 |
| 8 | Primary | −45 | 60 | R | 50 | 16.7 |
| | Drying | | | | | |
| 9 | Primary | −45 | 420 | H | 50 | 23.7 |
| | Drying | | | | | |
| 10 | Primary | −30 | 180 | R | 600 | 26.7 |
| | Drying | | | | | |
| 11 | Primary | −45 | 60 | R | 600 | 27.7 |
| | Drying | | | | | |
| 12 | Primary | −18 | 120 | R | 600 | 29.7 |
| | Drying | | | | | |
| 13 | Primary | −42 | 60 | R | 600 | 30.7 |
| | Drying | | | | | |
| 14 | Primary | −18 | 120 | R | 600 | 32.7 |
| | Drying | | | | | |
| 15 | Primary | −42 | 60 | R | 600 | 33.7 |
| | Drying | | | | | |
| 16 | Primary | −18 | 120 | R | 600 | 35.7 |
| | Drying | | | | | |
| 17 | Primary | −42 | 60 | R | 600 | 36.7 |
| | Drying | | | | | |
| 18 | Primary | −18 | 120 | R | 600 | 38.7 |
| | Drying | | | | | |
| 19 | Primary | −42 | 60 | R | 600 | 39.7 |
| | Drying | | | | | |
| 20 | Primary | −18 | 120 | R | 600 | 41.7 |
| | Drying | | | | | |
| 21 | Primary | −42 | 60 | R | 600 | 42.7 |
| | Drying | | | | | |
| 22 | Primary | −18 | 120 | R | 600 | 44.7 |
| | Drying | | | | | |
| 23 | Primary | −42 | 60 | R | 600 | 45.7 |
| | Drying | | | | | |
| 24 | Primary | −18 | 120 | R | 600 | 47.7 |
| | Drying | | | | | |
| 25 | Primary | −42 | 60 | R | 600 | 48.7 |
| | Drying | | | | | |
| 26 | Primary | −18 | 120 | R | 600 | 50.7 |
| | Drying | | | | | |
| 27 | Primary | −42 | 60 | R | 600 | 51.7 |
| | Drying | | | | | |
| 28 | Primary | −18 | 120 | R | 600 | 53.7 |
| | Drying | | | | | |
| 29 | Primary | −18 | 680 | H | 600 | 65.0 |
| | Drying | | | | | |
| 30 | Primary | −42 | 60 | R | 800 | 66.0 |
| | Drying | | | | | |
| 31 | Primary | −42 | 300 | H | 800 | 71.0 |
| | Drying | | | | | |

The product was evaluated for water content, Solvent content and reconstitution time. Results are tabulated in table 17.

As is

TABLE 17
Analytical results of lyophilizate:

| | | Solvent Content | | |
|---|---|---|---|---|
| | Water Content | Acetone | TBA | Reconstitution |
| Strength | (% w/w) | ppm | ppm | time (sec) |
| 500 mg/vial | 6.43 | 1235 | 652 | 35 |
| | 6.41 | 1026 | 626 | |
| | 6.59 | 1067 | 605 | |
| 1 g/vial | 6.43 | 1180 | 831 | 35 |
| | 6.41 | 1055 | 659 | |
| | 6.57 | 992 | 651 | |
| 2 g/vial | 6.51 | 1263 | 730 | 20 |
| | 6.57 | 893 | 748 | |
| | 6.51 | 899 | 507 | |

evident from the above results, the formation of monohydrate and the reconstitution time are consistent even at different strengths.

The procedures described herein are adapted to prepare other compositions described below:

### Example-2

| S. No | Ingredients | Quantity mg/Vial |
|---|---|---|
| 1. | Cyclophosphamide | 2000 |
| 2. | Acetonitrile:Water (30:70) | Q.s to 16.0 mL |

Acetonitrile and water were mixed and cooled to less than 10° C. (Temperature range: −10 to 10° C.). Drug was added while temperature was maintained in the range mentioned. The solution was filtered and filled in vials to target fill volume and freeze dried.

### Example-3

| S. No | Ingredients | Quantity mg/Vial |
|---|---|---|
| 1. | Cyclophosphamide | 2000 |
| 2. | Acetonitrile:Water (10:90) | Q.s to 80 mL |

Acetonitrile and water were mixed and cooled to less than 10° C. (Temperature range: −10 to 10° C.). Drug was added while temperature was maintained in the range mentioned. The solution was filtered and filled in vials to target fill volume and freeze dried.

### Example-4

| S. No | Ingredients | Quantity mg/Vial |
|---|---|---|
| 1. | Cyclophosphamide | 2000 |
| 2. | Acetone:Water (30:70) | Q.s to 55.5 mL |

Acetone and water were mixed and cooled to less than 10° C. (Temperature range: −10 to 10° C.). Drug was added while temperature was maintained in the range mentioned. The solution was filtered and filled in vials to target fill volume and freeze dried.

### Example-5

| S. No | Ingredients | Quantity mg/Vial |
|---|---|---|
| 1. | Cyclophosphamide | 2000 |
| 2. | Acetone:Tertiary butanol:Water | Q.s to 55.5 mL |
| | (15:15:30) | |

Acetone, Tertiary butanol and water were mixed and cooled to less than 10° C. (Temperature range: −10 to 10° C.). Drug was added while temperature was maintained in the range mentioned. The solution was filtered and filled in vials to target fill volume and freeze dried.

### Example-6

| S. No | Ingredients | Quantity mg/Vial |
|---|---|---|
| 1. | Cyclophosphamide | 2000 |
| 2. | Acetonitrile:Tertiary butanol:Water | Q.s to 16.0 mL |
| | (30:10:60) | |

Acetonitrile, Tertiary butanol and water were mixed and cooled to less than 10° C. (Temperature range: −10 to 10° C.). Drug was added while temperature was maintained in the range mentioned. The solution was filtered and filled in vials to target fill volume and freeze dried.

### Example-7

| S. No | Ingredients | Quantity mg/Vial |
|---|---|---|
| 1. | Cyclophosphamide | 2000 |
| 2. | Acetonitrile:Tertiary butanol:Water | Q.s to 24.0 mL |
| | (20:20:60) | |

Acetonitrile, Tertiary butanol and water were mixed and cooled to less than 10° C. (Temperature range: −10 to 10° C.). Drug was added while temperature was maintained in the range mentioned. The solution was filtered and filled in vials to target fill volume and freeze dried.

### Example-8

| S. No | Ingredients | Quantity mg/Vial |
|---|---|---|
| 1. | Cyclophosphamide | 2000 |
| 2. | Acetonitrile:Tertiary butanol:Water | Q.s to 36.0 mL |
| | (5:50:45) | |

Acetonitrile, Tertiary butanol and water were mixed and cooled to less than 10° C. (Temperature range: −10 to 10° C.). Drug was added while temperature was maintained in the range mentioned. The solution was filtered and filled in vials to target fill volume and freeze dried.

### Example-9

| S. No | Ingredients | Quantity mg/Vial |
|---|---|---|
| 1. | Cyclophosphamide | 500 |
| 2. | Acetone | 1 mL |
| 3. | Tertiary butanol | 0.75 mL |

Cyclophosphamide was dissolved in cool acetone (Temperature range: −10 to 10° C.). Tertiary butanol was added with continuous mixing. The solution was filtered and filled in vials to target fill volume and freeze dried.

### Example-10

| S. No | Ingredients | Quantity mg/Vial |
|---|---|---|
| 1. | Cyclophosphamide | 500 |
| 2. | Acetone | 1 mL |
| 3. | Tertiary butanol | 0.75 mL |
| 4. | Water | 0.3 mL |

Cyclophosphamide was dissolved in cool acetone (Temperature range: −10 to 10° C.). Tertiary butanol and water were added with continuous mixing. The solution was filtered and filled in vials to target fill volume and freeze dried.

### Example-11

| S. No | Ingredients | Quantity mg/Vial |
|---|---|---|
| 1. | Cyclophosphamide | 1000 |
| 2. | Acetone | 2 mL |
| 3. | Tertiary butanol | 1.5 mL |
| 4. | Water | 0.6 mL |

Cyclophosphamide was dissolved in cool acetone (Temperature range: −10 to 10° C.). Tertiary butanol and water were added with continuous mixing. The solution was filtered and filled in vials to target fill volume and freeze dried.

### Example-12

| S. No | Ingredients | Quantity mg/Vial |
|---|---|---|
| 1. | Cyclophosphamide | 1000 |
| 2. | Acetone | 2 mL |
| 3. | Tertiary butanol | 1.5 mL |

Cyclophosphamide was dissolved in cool acetone (Temperature range: −10 to 10° C.). Tertiary butanol was added with continuous mixing. The solution was filtered and filled in vials to target fill volume and freeze dried.

### Example-13

| S. No | Ingredients | Quantity mg/Vial |
|---|---|---|
| 1. | Cyclophosphamide | 2000 |
| 2. | Acetone | 4 mL |
| 3. | Tertiary butanol | 3 mL |

Cyclophosphamide was dissolved in cool acetone (Temperature range: −10 to 10° C.). Tertiary butanol was added with continuous mixing. The solution was filtered and filled in vials to target fill volume and freeze dried.

### Example-14

| S. No | Ingredients | Quantity mg/Vial |
|---|---|---|
| 1. | Cyclophosphamide | 2000 |
| 2. | Acetone | 4 mL |
| 3. | Tertiary butanol | 3 mL |
| 4. | Water | 1.2 mL |

Cyclophosphamide was dissolved in cool acetone (Temperature range: −10 to 10° C.). Tertiary butanol and water were added with continuous mixing. The solution was filtered and filled in vials to target fill volume and freeze dried.

### Example-15

Lyophilization experiments were performed using solvents that had lower vapour pressure than water for example: Dimethyl acetamide and Dimethylsulfoxide. It was found that the use of these solvents did not result in stable lyophilizate.

Composition:

| S. No | Ingredients | Quantity mg/Vial |
|---|---|---|
| 1. | Cyclophosphamide | 2000 |
| 2. | Dimethyl acetamide | 4 mL |
| 3. | Water | 0.5 mL |

Cyclophosphamide was dissolved in cool Dimethylacetamide (Temperature range: −10 to 10° C.) and water was added to the solution and mixed. The solution was filtered and filled in vials to target fill volume and freeze dried.

### Example-16

| S. No | Ingredients | Quantity mg/Vial |
|---|---|---|
| 1. | Cyclophosphamide | 2000 |
| 2. | Acetonitrile | 5.88 mL |
| 3. | Water | 2.52 mL |

The solvent mixture was prepared by taking acetonitrile and water in a mixing vessel and the solution was cooled to 2-8° C. Cyclophosphamide was added to 80% of solvent mixture and stirred well, until the drug is completely dissolved at 2-8° C. Volume was made up to 100% with solvent mixture and the bulk solution was filled into vials for lyophilization. The lyophilization cycle parameters are mentioned in below table 18:

TABLE 18
Lyophilization Cycle

| | Lyophilization | Temp | Process Time | | Vacuum | Cum |
|---|---|---|---|---|---|---|
| Step No | Step | (° C.) | (min) | Ramp/Hold | mtorr | Time (hrs) |
| 1 | Freezing | −10 | 10 | H | — | 0.166 |
| 2 | Freezing | −50 | 60 | R | — | 1.166 |
| 3 | Freezing | −50 | 400 | H | — | 7.83 |
| 4 | Freezing | −12 | 150 | R | — | 8.666 |
| 5 | Freezing | −12 | 240 | H | — | 12.66 |
| 6 | Freezing | −50 | 150 | R | — | 15.166 |
| 7 | Freezing | −50 | 200 | H | — | 18.50 |
| 8 | Primary Drying | −50 | 60 | R | 50 | 19.50 |
| 9 | Primary Drying | −50 | 180 | H | 50 | 22.50 |
| 10 | Primary Drying | −30 | 120 | R | 600 | 24.50 |
| 11 | Primary Drying | −50 | 60 | R | 600 | 25.50 |
| 12 | Primary Drying | −30 | 120 | R | 600 | 27.50 |
| 13 | Primary Drying | −50 | 60 | R | 600 | 28.50 |
| 14 | Primary Drying | −30 | 120 | R | 600 | 30.50 |
| 15 | Primary Drying | −50 | 60 | R | 600 | 31.50 |
| 16 | Primary Drying | −30 | 120 | R | 600 | 33.50 |
| 17 | Primary Drying | −30 | 180 | H | 600 | 36.50 |
| 18 | Primary Drying | −30 | 960 | H | 600 | 52.50 |
| 19 | Primary Drying | −30 | 120 | H | 350 | 54.50 |

Results: Water content (%) was 5.8% and Solvent Content (ppm) was 2500.

### Example 17

| | | |
|---|---|---|
| S. No | Ingredients | Quantity mg/Vial |

| | | | |
|---|---|---|---|
| 1. | Cyclophosphamide | 2000 | mg |
| 2. | Mannitol | 1750 | mg |
| 3. | Tertiary Butanol | 11 | mL |
| 4. | Water | 15 | mL |

Initially mannitol was dissolved in required quantity of water in a mixing vessel and cooled to 10° C.-15° C. In another mixing vessel Cyclophosphamide was dissolved in 80% of required quantity of Tertiary butanol. Mannitol solution was added to the drug solution and stirred at 10° C.-15° C. to get a uniform solution. Volume was made up to 100% with Tertiary butanol and the bulk solution was filled into vials for lyophilisation. The sample was then freeze dried.

### Example 18

| | | |
|---|---|---|
| S. No | Ingredients | Quantity mg/Vial |

| | | | |
|---|---|---|---|
| 1. | Cyclophosphamide | 2000 | mg |
| 2. | Mannitol | 1750 | mg |
| 3. | Acetone | 11 | mL |
| 4. | Water | 15 | mL |

Initially mannitol was dissolved in required quantity of water in a mixing vessel and was cooled to 10° C.-15° C. In another mixing vessel Cyclophosphamide was dissolved in 80% of required quantity of Acetone. Mannitol solution was added to the drug solution and stirred at 10° C.-15° C. to get a uniform solution. Volume was made up to 100% with Acetone and the bulk solution was filled into vials for lyophilisation. The sample was then freeze dried.

### Example 19

| | | |
|---|---|---|
| S. No | Ingredients | Quantity mg/Vial |

| | | | |
|---|---|---|---|
| 1. | Cyclophosphamide | 2000 | mg |
| 2. | Mannitol | 1750 | mg |
| 3. | Acetonitrile | 8 | mL |
| 4. | Water | 15 | mL |

Initially mannitol was dissolved in required quantity of water in a mixing vessel and was cooled to 10° C.-15° C. In another mixing vessel Cyclophosphamide was dissolved in 80% of required quantity of Acetonitrile. Mannitol solution was added to the drug solution and stirred at 10° C.-15° C. to get a uniform solution. Volume was made up to 100% with Acetonitrile and the bulk solution was filled into vials for lyophilisation. The sample was then freeze dried.

### Example 20

| | | |
|---|---|---|
| S. No | Ingredients | Quantity mg/Vial |

| | | | |
|---|---|---|---|
| 1. | Cyclophosphamide | 2000 | mg |
| 2. | Sodium Chloride | 900 | mg |
| 3. | Acetone | 10 | mL |
| 4. | Acetonitrile | 10 | mL |
| 5. | Water | 24 | mL |

Initially sodium chloride was dissolved in required quantity of water in a mixing vessel. In another mixing vessel solvent mixture was prepared by taking acetone and acetonitrile and the solution was cooled to 2-8° C. Cyclophosphamide was added to 80% of solvent mixture and stirred well, until the drug is completely dissolved at 2-8° C. Sodium chloride solution was added to solvent mixture and stirred to get a uniform solution. Volume was made up to 100% with solvent mixture and the bulk solution was filled into vials for lyophilisation. The sample was then freeze dried.

### Example 21

| | | |
|---|---|---|
| S. No | Ingredients | Quantity mg/Vial |

| | | | |
|---|---|---|---|
| 1. | Cyclophosphamide | 2000 | mg |
| 2. | Sodium Chloride | 900 | mg |
| 3. | Acetone | 9 | mL |
| 4. | Water | 7 | mL |

Initially sodium chloride was dissolved in required quantity of water in a mixing vessel. In another mixing vessel 80% of required quantity of Acetone was taken cooled to 2-8° C. Cyclophosphamide was added to above solvent and stirred well, until the drug is completely dissolved at 2-8° C. Sodium chloride solution was added to drug solution and stirred to get uniform solution. Volume was made up to 100% with Acetone and the bulk solution was filled into vials for lyophilisation. The sample was then freeze dried.

1-40. (canceled)

41. A composition comprising lyophilized cyclophosphamide monohydrate where in lyophilization process is carried out in presence of solvents without a rehydration step.

42. A composition comprising lyophilized cyclophosphamide monohydrate where in lyophil ization is carried out in presence of solvent or mixtures of solvents selected from group comprising of Acetone, Acetonitrile, Tertiary butanol, Methanol, Ethanol, Isopropyl alcohol, Ethylacetate, Toluene, Propyl acetate, Nitromethane, 1,4-Dioxane, Heptane, Methylcyclohexane, Formic acid, isopropyl acetate, 1,2-Dimethoxyethane, 1,1,2-Trichloroethene, Methylethyl ketone, Cyclohexane, Ethyl formate, Hexane, Tetrahydrofuran, Chloroform, Methyl acetate, 1,2-Dichloroethene, tert-Butylmethyl ether, Dichloromethane, Pentane, Ethyl ether, Sulfalone, Ethyleneglycol, Formamide, Tetralin, N-Methylpyrrolidone, Dimethyl sulfoxide, N,N-Dimethylacetamide, 1-Pentanol, 3-Methyl-1-butanol, Anisole, N,N-Dimethylformamide, 2-Ethoxyethanol, 1-Butanol, 2-Methoxyethanol, Cumene, Butyl acetate, 2-Methyl-1-propanol, Chlorobenzene, Acetic acid, 2-Butanol, Isobutyl acetate, 1-Propanol, Pyridine, Methylbutyl ketone, Methyl isobutyl ketone, Xylene, Dimethyl carbonate, Hexafluoroacetone, Chlorobutanol, Dimethylsulfone, carbon tetrachloride and tetrahydropyran.

43. The composition according to claim 42, wherein lyophilization is carried out using a mixture of solvents selected from Acetone and Acetonitrile, Acetone and Tertiary butanol, Acetonitrile and Tertiary butanol, Isopropyl alcohol and Tertiary butanol, Methanol and Tertiary butanol, Ethanol and Tertiary butanol, Methanol and Isopropyl alcohol, Ethanol and Isopropyl alcohol, Acetonitrile and Isopropyl alcohol.

44. The composition according to claim 43, wherein lyophilization is carried out using a mixture of solvents and water.

45. The composition according to claim 44, wherein lyophilization is carried out using a mixture acetone, tertiary butanol and water.

46. The composition according to claim 44, wherein lyophilization is carried out using a solvent or mixture of solvents comprising at least 25% v/v of the total composition.

47. The composition of claim 46 wherein the lyophilisation process further comprises water between 0.5% to 60% v/v of the total composition

48. A lyophilization process for the manufacture of Cyclophosphamide injection comprising the following steps:
(a) dissolving Cyclophosphamide in solvent or mixture of solvents for freeze drying at temperature below 15° C.
(b) filling the vials with the Cyclophosphamide solution of step (a)
(c) loading of filled vials into precooled lyophilizer maintained below 10° C.
(d) cooling or freezing the vials
(e) drying the product by removing the solvent and excess water under vacuum

49. The process of claim 48, wherein freezing is performed at temperatures below −12° C.

50. The process according to claim 48, wherein drying is performed at temperatures between −50° C. and +5° C.

51. The process according to claim 48, wherein vacuum used for drying steps is between 10 mtorr and 1500 mbar.

52. The process of claim 48, which further comprises of at least one negative temperature rate step.

53. The process of claim 48, wherein the drying step comprises of at least one annealing step.

54. The process according to claim 48, wherein the temperature used for drying is less than or equal to 5° C.
